(19) United States
(12) Patent Application Publication
(10) Pub. No.: US 2025/0189684 A1
(43) Pub. Date: Jun. 12, 2025

(54) REALTIME HAZARDS VISUALIZATION

(71) Applicant: The Center for Headache, Spine, and Pain Medicine PLLC, Dallas, TX (US)

(72) Inventors: Edward Casteel Milner, Dallas, TX (US); Ezekiel Fink, Dallas, TX (US); Krag Browder, Colleyville, TX (US); Benjamin Michael Evans, Plano, TX (US); Faisal Sunesara, Houston, TX (US); San Luong, Garland, TX (US); Kael Madar, Southlake, TX (US); Vishakh Nair, San Ramon, CA (US); Ashwin Somasundaram, Cypress, TX (US); Jim Moore, Dallas, TX (US); Hansika Sundaresan, Dallas, TX (US)

(73) Assignee: The Center for Headache, Spine, and Pain Medicine PLLC, Dallas, TX (US)

(21) Appl. No.: 18/847,171

(22) PCT Filed: Mar. 14, 2023
(86) PCT No.: PCT/US2023/064339

§ 371 Date: Sep. 13, 2024

Related U.S. Application Data
(60) Provisional application No. 63/490,159, filed on Mar. 14, 2023.
(60) Provisional application No. 63/362,960, filed on Apr. 13, 2022.
(60) Provisional application No. 63/269,300, filed on Mar. 14, 2022.

(51) Int. Cl. G01T 1/169; A61B 6/46; G01T 7/00; G08B 21/12; G16H 40/20

(52) U.S. Cl. CPC G01T 1/169; A61B 6/461; G01T 7/00; G08B 21/12; G16H 40/20

(57) ABSTRACT

Apparatus and associated methods relate to dynamically monitoring radiation exposure of sensitive objects. In an illustrative example, a dynamic hazard visualization system may provide real time radiation exposure monitoring for medical personnel in an operation room. A dynamic hazard visualization system (e.g., DHVS), for example, may include a data store comprising a plurality of predetermined radiation intensity models. For example, the DHVS may receive at least one image received from a camera installed in the operation room. Based on the received image and one or more predetermined radiation intensity models, the DHVS may generate an inter-object properties data structure (e.g., IOPD) of the operation room. The DHVS may use the IOPS to generate, in real time, an instantaneous radiation intensity profile based on a dynamic geometry generated based on updated images received from the camera. Various embodiments may advantageously monitor radiation exposure of the medical personnel in real time.

### CROSS-REFERENCE TO RELATED APPLICATIONS

This application claims the benefit of U.S. Provisional Application Ser. No. 63/269,300, titled “Realtime Hazards Visualization,” filed by Edward Casteel Milner, et al., on Mar. 14, 2022, U.S. Provisional Application Ser. No. 63/362,960, titled “Dynamic Registration of Hazard Visualization,” filed by Ben Evans, et al., on Apr. 13, 2022, and U.S. Provisional Application Ser. No. 63/490,159, titled “Realtime Hazards Visualization,” filed by Edward Casteel Milner, et al., on Mar. 14, 2023.

This application incorporates the entire contents of the foregoing application(s) herein by reference.

### TECHNICAL FIELD

Various embodiments relate generally to visualization of hazards.

### BACKGROUND

Medical personnel safety is an important aspect of an operation room. For example, doctors, nurses, and other medical staff may be exposed to various hazards in an operation. One major concern is exposure to ionizing radiation. For example, the ionizing radiation may be used in medical procedures such as X-rays and CT scans. While these procedures are generally safe for patients, they can pose risks to medical personnel who work in the same room as the equipment repeatedly. In addition, medical personnel may also be exposed to other types of hazardous emissions, such as surgical smoke, anesthesia gases, and chemicals used in cleaning and disinfecting equipment. Therefore, safety measures are important for medical facilities to protect the health and well-being of the medical personnel. Some safety measures, for example, may include regular training on safety procedures. Some safety measures, for example, may include the use of protective equipment to limit exposure to hazardous emissions generated during medical procedures.

Various environments may include equipment that generates emissions. As an illustrative example, radiography may be used, by way of example and not limitation, in industrial, commercial, research, and/or medical fields. For example, a physician may use a medical imaging machine to emit electromagnetic radiation for diagnosis, therapy, and/or procedural guidance.

In order to protect medical personnel from radiation exposure of the medical imaging machine, various safety measures are employed, such as the use of radiation shields, personal protective equipment, and radiation monitoring devices. Some hazardous radiation exposure may cause acute and long-term health effects, including cancer, cataracts, and genetic mutations.

### SUMMARY

Apparatus and associated methods relate to dynamically monitoring radiation exposure of sensitive objects. In an illustrative example, a dynamic hazard visualization system may provide real time radiation exposure monitoring for medical personnel in an operation room. A dynamic hazard visualization system (DHVS), for example, may include a data store comprising a plurality of predetermined radiation intensity models. For example, the DHVS may receive at least one image received from a camera installed in the operation room. Based on the received image and one or more predetermined radiation intensity models, the DHVS may generate an inter-object properties data structure (IOPD) of the operation room. The DHVS may use the IOPS to generate, in real time, an instantaneous radiation intensity profile based on a dynamic geometry generated based on updated images received from the camera. Various embodiments may advantageously monitor radiation exposure of the medical personnel in real time.

Apparatus and associated methods relate to dynamic hazard visualization. In an illustrative example, a visualization model may be generated based on a target hazard emitter operating configuration. The visualization model may, for example, be applied to geometry of the target hazard emitter and/or (expected, target) surrounding objects to generate a visualization pattern. The visualization pattern may, for example, be provided to a visualization generator. The visualization generator may generate hazard indicia visible, for example, to humans within at least one danger zone around the target hazard emitter. Various embodiments may advantageously enable personnel to reduce exposure to hazardous emissions in an environment.

Various embodiments may achieve one or more advantages. For example, some embodiments may include a computer vision engine to advantageously identify a dynamic geometry, in real time, of positions and orientation of objects in the operation room. Some embodiments, for example, may include an alert generation engine to advantageously generate one or more visual indicium to display real time radiation hazards in the operation room. For example, some embodiments may include non-electronic orientation tags to advantageously provide orientation information in an image of the objects in the operation room. Some embodiments, for example, may generate a visualization overlay on the image captured by the camera to advantageously provide an augmented reality display. For example, some embodiments may advantageously generate suggestions to the medical personnel to minimize hazardous radiation exposure. Some embodiments, for example, may include pre-calculated radiation models to advantageously shorten a calculation time to generate a radiation mapping of the operation room.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary dynamic hazard visualization system (DHVS) employed in an illustrative use-case scenario.

FIG. 2 depicts a top view of an exemplary DHVS in an illustrative use-case scenario.

FIG. 3 depicts a block diagram of an exemplary DHVS.

FIG. 4 depicts a block diagram of an exemplary interactive DHVS.

FIG. 5A depicts a block diagram of an exemplary visualization engine 145 and a visualization model.

FIG. 5B depicts an exemplary embodiment including orientation tags configured to provide additional orientation information for generating the visualization model as described with reference to FIG. 5A.

FIG. 6 depicts an exemplary method of generating a visualization model.

FIG. 7 depicts an exemplary method of generating a visualization.

FIG. 8 depicts an exemplary method of using N predetermined emission models to generate a transformed radiation model (TRM).

Like reference symbols in the various drawings indicate like elements.

### DETAILED DESCRIPTION OF ILLUSTRATIVE EMBODIMENTS

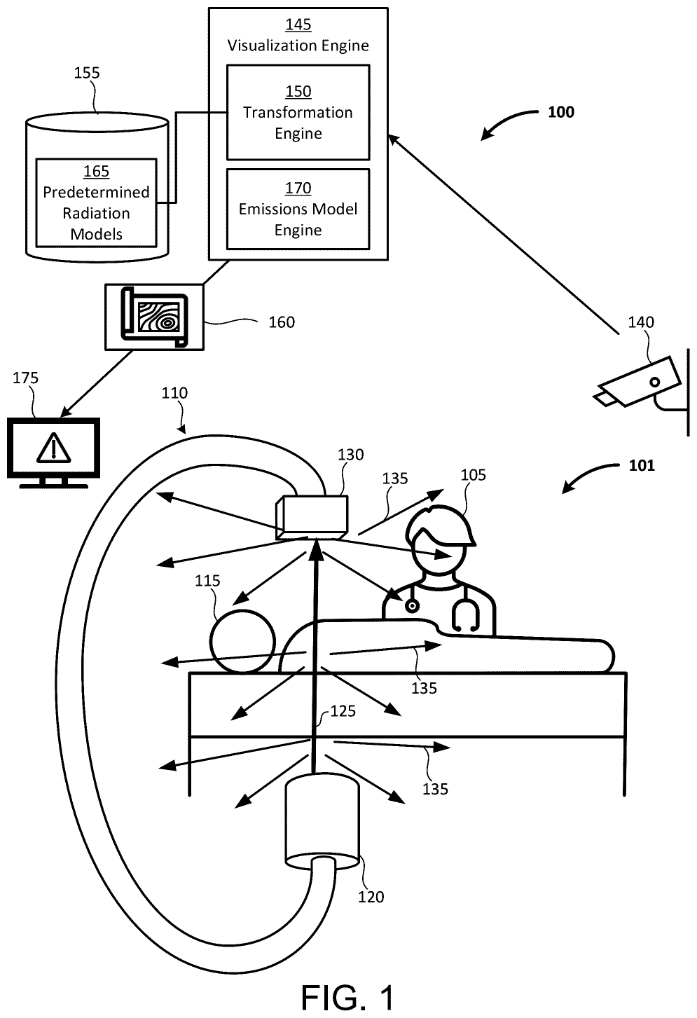

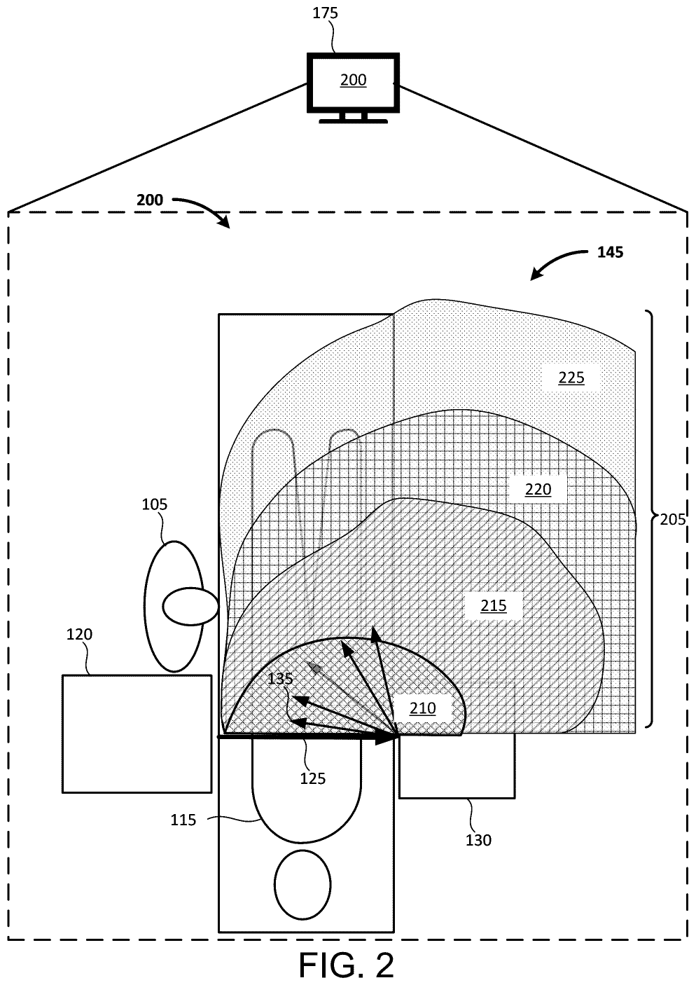

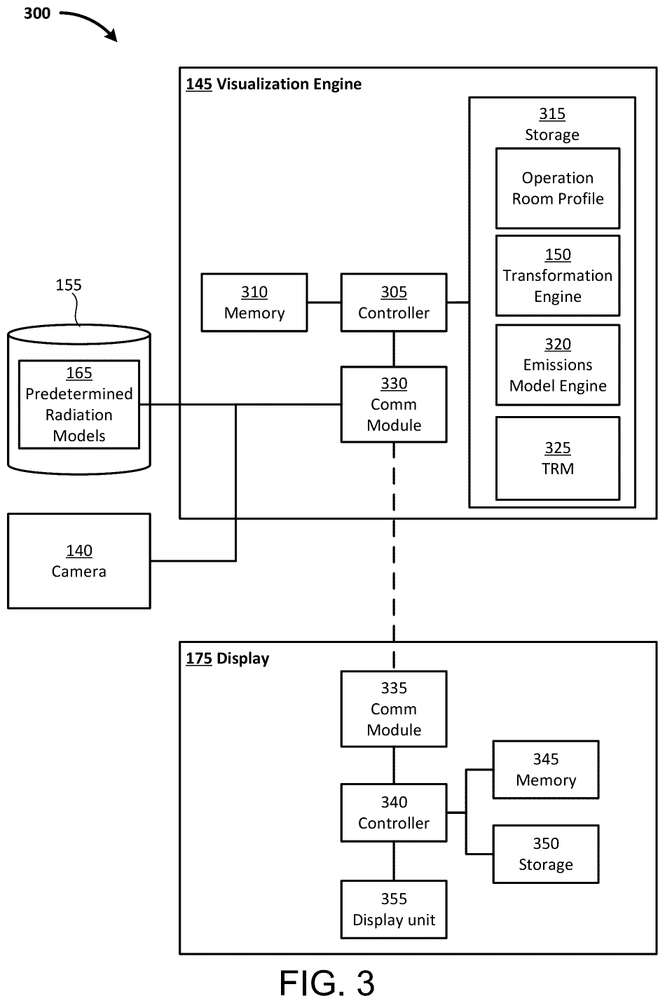

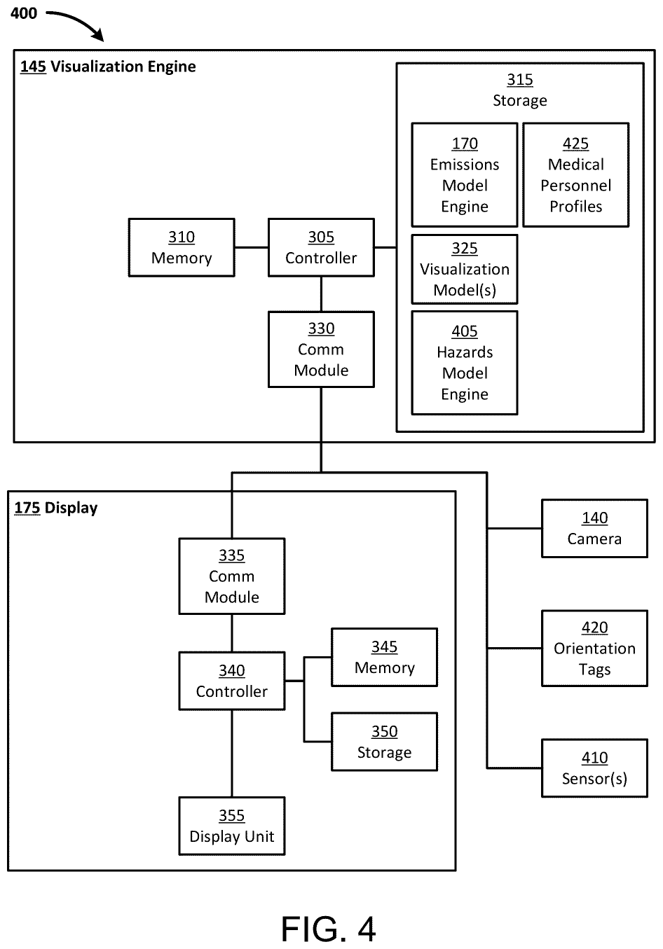

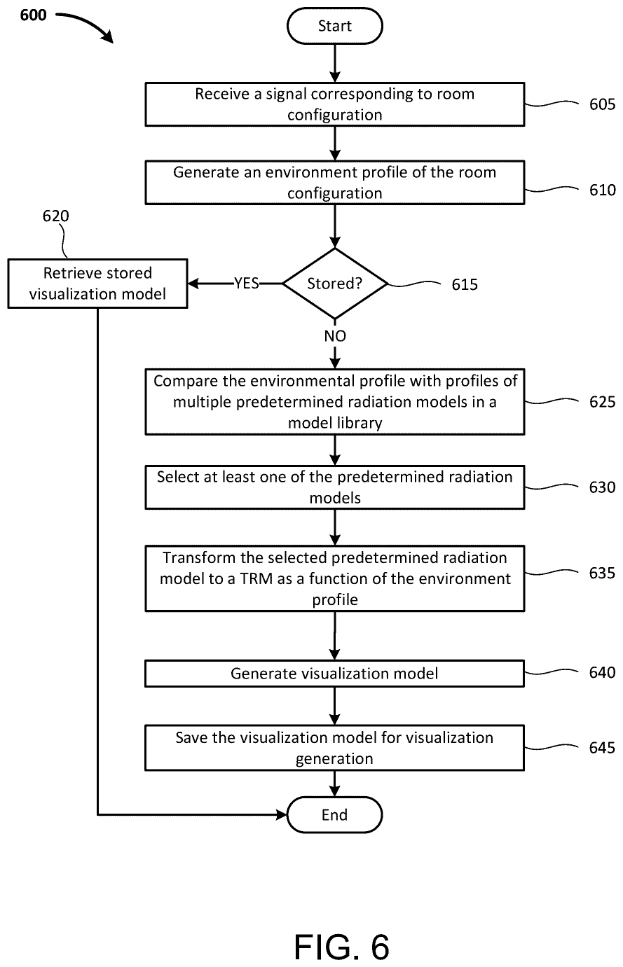

To aid understanding, this document is organized as follows. First, to help introduce discussion of various embodiments, a dynamic hazard visualization system (DHVS) system is introduced with reference to FIGS. 1-2. Second, that introduction leads into a description with reference to FIGS. 3-4 of some exemplary embodiments of DHVSs. Third, with reference to FIGS. 6-8, exemplary methods related to dynamic hazard visualization are described. Finally, the document discusses further embodiments, exemplary applications and aspects relating to dynamic hazard visualization.

FIG. 1 depicts an exemplary dynamic hazard visualization system (DHVS 100) employed in an illustrative use-case scenario. In an illustrative operating environment (e.g., a hospital operating room 101), a user 105 (e.g., a physician) is operating a C-arm radiography machine 110 to image a patient 115. As depicted, the c-arm radiography machine 110 includes an emitter 120. The emitter 120 generates and emits a beam 125 of electromagnetic radiation. The beam 125 impinges upon a receiver 130. As the beam travels to the receiver 130, it may travel through various materials, such as the operating table, the patient 115, and/or the receiver 130. At least some of the beam 125 is reflected as it passes through one or more of these materials, as scattered radiation 135. The scattered radiation 135 may, for example, strike the user 105.

The scattered radiation 135 may, for example, strike other objects and further scatter. The scattered radiation 135 may, for example, expose the user 105, other users, and/or standers-by to hazardous radiations. For example, in the case of a surgeon, the user 105 may be exposed each time to relatively low doses (e.g., below a ‘safety threshold’) of the scattered radiation 135. Over time (e.g., using the c-arm radiography machine 110 for guidance during medical operations during multiple procedures each day), the user 105 may be exposed to dangerous amounts of the scattered radiation 135. In some examples, the c-arm radiography machine 110 may be oriented in a different orientation. For example, instead of traversing vertically as shown in FIG. 1, the beam 125 may be transmitted in a direction parallel to a direction towards the user 105. In this case, for example, the user 105 may be exposed to the scattered radiation at a higher rate than the orientation of the c-arm radiography machine 110 as shown in FIG. 1.

The DHVS 100 includes a camera 140. For example, the camera 140 may be a digital camera. For example, the camera 140 may be a mobile phone camera. For example, the camera may be a three-dimensional (3D) camera. The camera 140 is operably coupled to a visualization engine 145. For example, the camera 140 may be physically (e.g., a local area network) coupled to the visualization engine 145. For example, the camera 140 may be wirelessly coupled to the visualization engine 145 via a communication network (e.g., a WIFI network, a Bluetooth network, a radio network). In some implementations, the camera 140 may be a wide angle camera that covers the whole operation room 101. In some implementations, the DHVS 100 may include more than one camera 140 to capture multiple images at the same time for analysis.

The visualization engine 145 includes a transformation engine 150 in this example. In some implementations, the visualization engine 145 may use the transformation engine 150 to generate a radiation exposure mapping based on one or more images captured by the camera 140. As shown, the DHVS 100 includes a data store 155. For example, the data store 155 may be operably coupled to the visualization engine 145. For example, the data store 155 may be remotely coupled to the visualization engine 145 via a network. The data store 155 includes predetermined radiation models 165. For example, the predetermined radiation models 165 may include radiation models of various configurations (e.g., orientation configurations, equipment configurations, operation room configurations). For example, the predetermined radiation models 165 may include predetermined radiation intensity distribution simulation data.

Based on the radiation models, the visualization engine 145 may, for example, generate a visualization model 160 for the hospital operating room 101. For example, the visualization model 160 may include properties and characteristics (e.g., width, height, intervals) of the hospital operating room 101 like a digital twin of the hospital operating room 101. For example, each of the predetermined radiation models 165 may be generated based on multiple predetermined configurations (e.g., 5, 10, 15, 100 configurations) with one or more predetermined simulation data models (e.g., Geometry and Tracking 4 (GEANT4) data model, Monte Carlo N-Particle Transport Code, Electron Gamma Shower (EGS) computer code system). For example, some of the predetermined radiation models 165 may include actual measurement data of radiation exposure collected from experiment in known configurations. For example, some of the predetermined radiation models 165 may be pre-calculated based on specific configurations.

In some implementations, the predetermined radiation models 165 may include a data object that links various configuration parameters (e.g., size of the hospital operating room hospital operating room 101, volume of the hospital operating room 101, type of equipment, number of person within the hospital operating room 101, type of the c-arm radiography machine 110, orientation of the 110, position of the c-arm radiography machine 110) to generate a visualization model 160. For example, the visualization model 160 may include an inter-object properties data structure. The inter-object properties data structure may include, for example, properties (e.g., dimension, material, reflection characteristics) of the hospital operating room 101, properties of scattering objects in the hospital operating room 101, orientation of a radiation source, (dynamic) positions of personnel in the hospital operating room 101, or a combination thereof.

In some implementations, the transformation engine 150 may select one or more of predetermined radiation models 165 to generate the visualization model 160. For example, the transformation engine 150 may select, based on the image captured from the camera 140, at least one of the predetermined radiation models 165 for generating the visualization model 160 for the hospital operating room 101. For example, the transformation engine 150 may select the one or more predetermined radiation models 165 based on a detected size and dimension of the hospital operating room 101. In some implementations, the transformation engine 150 may select the predetermined radiation models 165 based on an operation profile. For example, the operation profile may include a type radiation source (e.g., the c-arm radiography machine 110, an X-ray machine, a MRI machine) used in the hospital operating room 101. For example, the transformation engine 150 may generate a transformed radiation model corresponding to a specific configuration of the hospital operating room 101. For example, the operation profile may indicate an initial state of the hospital operating room 101 as identified based on images captured by the camera, for example, at an initial stage of an operation.

In some implementations, the transformation engine 150 may transform the predetermined radiation models 165 based on detected dimension and/or orientation of the hospital operating room 101. For example, the transformation engine 150 may translate, rotate, and/or scale a predetermined radiation model 165 to fit the hospital operating room 101. For example, the transformation engine 150 may extrapolate the selected predetermined radiation models 165 based on scattering objects in the hospital operating room 101 and dimensions detected in the hospital operating room 101.

In this example, the visualization engine includes an emission model engine 170 configured to generate an instantaneous radiation intensity profile for the operation room as a function of the transformed radiation model. For example, based on the transformed radiation models, for example, the emission model engine 170 may apply a real-time configuration of the hospital operating room 101 to the transformed radiation models to generate the visualization model 160.

In this example, the DHVS 100 includes a display 175. For example, the display 175 may include a monitor. For example, the display 175 may include a television. For example, the display 175 may include a projector and a projector screen. In some implementations, the display 175 may visually display the visualization model 160 generated by the visualization engine 145. For example, the visual display may, for example, be color coded (e.g., as a ‘heat map’ of hazard). The visual display may, for example, include alphanumeric characters (e.g., measurements such as distance, intensity; warning labels; information labels). In some embodiments, the visual display may, for example, include images (e.g., icons, pictorial labels). Accordingly, the user 105 may, for example, advantageously determine ‘at-a-glance’ where to position themself to reduce hazard exposure. The visual display may, for example, induce the user 105 to instinctively reduce hazard exposure (e.g., by moving away from ‘red areas’ of hazard towards ‘green areas’ of reduced hazard).

In this example, the display 175 may also display an alert for the user 105. For example, the visualization engine 145 may be configured to determine an accumulative exposure for the user 105. For example, the visualization engine 145 may further include an alert generation engine (not shown in FIG. 1) configured to generate an indicium (e.g., a visual alert, a sound alert) communication a real time radiation hazards of the user 105 based on, for example, the instantaneous radiation intensity profile. For example, for each of the medical personnel in the hospital operating room 101, the visualization engine 145 may independently determine an accumulated exposure. In some implementations, the visualization engine 145 may determine the accumulated exposure based on historical radiation mappings and historical position and/or orientations of the user 105. For example, the visualization engine 145 may display an alert to the medical personnel when his/her accumulated exposure is above a predetermined threshold. In some implementations, the display 175 may include an augmented reality display of suggested action for the user 105 based on a relative position of the object to reduce radiation exposure on the user 105.

In some implementations, the DHVS 100 may include the camera 140 operably coupled to the visualization engine 145 and the predetermined radiation models 165. For example, the predetermined radiation models 165 may be configured to generate an inter-object properties data structure (e.g., the visualization model 160 by the transformation engine 150) as a function of one or more images of an operation room captured by the camera 140.

In some implementations, the inter-object properties data structure may include a position and orientation of a C-arm radiography machine, and a relative location of at least one target radiation-scattering object (e.g., medical personnel, scattering objects in the hospital operating room 101, an operation table in the hospital operating room 101) and at least one target radiation-sensitive object (e.g., the user 105, other medical personnel). In some implementations, the DHVS 100 may also include the emission model engine configured to generate an instantaneous radiation intensity profile for the operation room as a function of the inter-object properties data structure. For example, based on the relative geometry of the C-arm radiography machine 110 and the at least one target radiation-scattering object and the at least one radiation-sensitive object in real time, and a selected at least one predetermined radiation intensity distribution simulation. For example, the DHVS 100 may further include an alert generation engine configured to generate a visual indicium including real time radiation hazards of the at least one radiation-sensitive object based on the instantaneous radiation intensity profile.

In some implementations, the transformation engine 150 may select predetermined radiation intensity distribution simulation data from a library of predetermined simulation data sets (e.g., the predetermined radiation models 165). For example, the transformation engine 150 may select the predetermined radiation intensity distribution simulation data based on an operation room profile generated as a function of an initial state of the inter-object properties data structure.

In some implementations, the transformation engine 150 may apply transformation operations to one or more selected predetermined radiation models 165. For example, the transformation operations may be used to match geometrical relationships of objects in the operation room. Accordingly, the visualization model 160 may generate advantageously in near real time without performing a simulation based on the operation profile. In some implementations, the predetermined radiation models 165 may include a predetermined visualization overlay from which the visual indicium of real-time radiation hazards is generated. As such, for example, the DHVS 100 may notify the user 105 in real time when an urgent radiation exposure concern is detected. In some implementations, the visual indicium also includes an augmented reality display of suggested action for the at least one radiation-sensitive object based on a relative position of the object to reduce radiation exposure on the object.

In some examples, the user 105 may wear a radiation badge (not shown). However, the radiation badge may be read at relatively long periods (e.g., days, weeks, months) and so may be read too late to provide useful feedback to the user 105 for reducing radiation exposure. In some examples, the radiation badges may include reading that depends on an orientation of the user. For example, the user 105 may wear the radiation badge in front of the body while working for a long period of time facing the c-arm radiography machine 110 with his/her back. In some implementations, the DHVS 100 may account for the orientation and position of the user 105 and generate alerts and measurements related to the user 105 in real time.

FIG. 2 depicts a top view 200 of an exemplary DHVS in an illustrative use-case scenario. In some implementations, the top view 200 may be displayed in the display 175. As an illustrative example, the top view 200 may show the hospital operating room 101. As shown, the user 105 is operating an emitter 120 to image the patient 115. The emitter 120 emits a beam 125 which strikes the receiver 130 and generates the scattered radiation 135. For example, images of the configuration shown in the top view 200 may be captured by the camera 140.

The top view 200 may be generated by the visualization engine 145 based on the captured image from the camera 140. In this example, the top view 200 includes a virtual overlay 205. For example, the virtual overlay 205 be generated based off of the image captured by the camera 140. In some embodiments, for example, the visualization model 160 may include the virtual overlay 205. For example, the virtual overlay 205 may, for example, be transferred to the display 175 to be displayed as an augmented reality image on top of the captured image by the camera 140. As shown, the virtual overlay 205 includes a first visual indicium 210, a second visual indicium 215, and a third visual indicium 220. The first visual indicium 210, the second visual indicium 215, and the third visual indicium 220 may, for example, correspond to relative hazard regions. For example, the first visual indicium 210 may indicate a space where the radiation intensity is extremely high. For example, the user 105 may avoid placing any part of his/her body within an area of the first visual indicium 210. For example, the second visual indicium 215 may indicate a space where the radiation intensity is high. For example, the user 105 may determine to only put, if necessary, to place his/her body within an area of the second visual indicium 215. For example, the third visual indicium 220 may indicate a space where the radiation intensity is medium. For example, the user 105 may desire to minimize exposure within an area of the second visual indicium 220 during an operation. In this example, a fourth visual indicia 225 is also displayed. For example, the fourth visual indicia 225 may include low radiation exposure areas in the operation room. In some implementations, the DHVS 100 may continuously detect radiation exposure of the user 105 while the user 105 is in any of the areas indicated by the visual indicia 210, 215, 220, 225.

In the depicted example, the display 175 may receive the visualization model 160, for example, from the visualization engine 145. The visualization engine 145 may, for example, generate the visualization based on configuration and/or geometry (e.g., expected, predetermined, dynamic) as discussed with reference to FIG. 1. As depicted, the visualization engine 145 is operably coupled to a camera 140 (e.g., camera). The visualization engine 145 may, for example, automatically generate a visualization based on feedback from the camera 140 (e.g., position of the emitter 120, position of the user 105, position of other objects in the hospital operation room 101). Accordingly, for example, the visual indicia 210, 215, 220, 225 may, for example, be (automatically, dynamically) generated in response to actual (e.g., real-time) conditions in the hospital operation room 101.

In some implementations, the predetermined radiation models 165 may include a predetermined visualization overlay for generating the visual indicia 210, 215, 220, 225. For example, the predetermined visualization overlay may include radiation intensity as a function of a geometry in the model. In some implementations, the visualization engine 145 may generate an augmented reality visualization based on the predetermined visualization overlay. For example, the augmented reality visualization may include suggested actions for the at least one radiation-sensitive object based on the instantaneous radiation intensity profile to reduce radiation exposure on the at least one radiation-sensitive object. In some examples, a sound alert may be generated.

FIG. 3 depicts a block diagram of an exemplary DHVS 300. In this example, the DHVS 300 includes the visualization engine 145. The visualization engine 145 includes a controller 305. The controller 305 may, by way of example and not limitation, include one or more processors (e.g., microprocessor). The controller 305 is operably coupled to a memory module 310 (e.g., random-access memory). The memory module 310 may, for example, include one or more physical and/or logical memory devices. The controller 305 is operably coupled to a storage module 315 (e.g., nonvolatile memory). The storage module 315 may, for example, include one or more physical and/or logical data storage devices.

In the depicted example, the storage module 315 includes the visualization engine 150 and the emissions model engine 170. For example, the transformation engine 150 may receive from the data store 155 one or more predetermined radiation models 165 based on an operation room profile 320. For example, the transformation engine 150 may generate the operation room profile 320 as a function of one or more images received from the camera 140 corresponding to the hospital operating room 101. For example, the predetermined radiation models 165 may include predetermined physics simulations and/or historical physics data corresponding to hazard emission geometry corresponding to a hazard emitter location. The transformation engine 150 may, for example, (cause the controller 305 to) generate one or more transformed radiation models (TRM 325). The TRM 325 may, for example, be stored in the storage module 315. The TRM 325 may, for example, include an inter-object properties data structure.

The emissions model engine 170 may, for example, generate visualization models (e.g., the visualization model 160) based on the TRM 325. The emissions model engine 170 may, for example, generate visualization models as a function of the TRM 325 and a composition and/or position of material(s) an emitted beam (e.g., x-ray) is determined and/or predicted to pass through on its way to a receiver (e.g., the receiver 130). In some embodiments, by way of example and not limitation, the emissions model engine 170 may, for example, generate visualization model(s) as a function of the emitted beam properties (e.g., shape, intensity, energy level). For example, the emissions model engine 170 may generate and/or select visualization model(s) based on a current setting(s) of an emitter (e.g., the emitter 120).

The controller 305 is operably coupled to a communication module 330 (e.g., input-output module). The communication module 330 may, for example, include wired communication hardware and/or software. The communication module 330 may, for example, include wireless communication hardware and/or software. As shown, the communication module 330 is connected to the camera 140, and the data store 155. In some embodiments, the data store 155 may be included in the visualization engine 145.

The communication module 330 is in communication with a communication module 335 of a display 175. The communication module 330 and the communication module 335 may, for example, be in wireless communication. The communication module 330 and the communication module 335 may, for example, be in wired communication. The communication module 330 and the communication module 335 may, for example, be in communication via a physical, non-transitory transfer medium.

The display 175 includes a controller 340 (e.g., one or more processors). The controller 340 is operably coupled to the communication module 335. The controller 340 is operably coupled to a memory module 345 (e.g., one or more storage devices). The controller 340 is operably coupled to a storage module 350 (e.g., one or more storage devices). In some embodiments, the memory module 345 and/or the storage module 350 may, for example, be omitted. The controller 340 is operably coupled to a display unit 355. The controller 305 may, for example, generate a visualization from a visualization model 160. The controller 340 may receive the visualization (e.g., via the communication module 330 and the communication module 335). The controller 340 may operate the display unit 355 to generate and display visual indicia (e.g., the visual indicia as described with reference to FIG. 2) as a function of the visualization.

In some embodiments, the display unit 355 may, for example, be integral with the display 175. The display unit 355 may be physically separated from but operably coupled to the display 175. In some embodiments, the visualization engine 145 and the display 175 may, for example, be integral to a single device. In some embodiments, the visualization engine 145 may be separated from the display 175 (e.g., in the same building, in the same room). In some embodiments, the visualization engine 145 may be remote from the display 175 (e.g., connected via a cloud, selectively connected).

FIG. 4 depicts a block diagram of an exemplary interactive DHVS 400. In this example, the storage module 315 of the visualization engine 145 further includes a hazards model engine 405. The hazards model engine 405 may, for example, generate a visualization as a function of (predetermined) hazard levels and/or geometries. For example, the emissions model engine 170 may generate a model of emissions (e.g., visualization model, 2D emissions model data, 3D emissions model data). The hazards model engine 405 may, for example, operate on the output of the emissions model engine 170 to generate a visualization model as a function of the output of the emissions model engine 170 and hazards data (e.g., predetermined, historical, calculated based on predetermined data). As an illustrative example, the hazards model engine 405 may operate on the output of the emissions model engine 170 (e.g., based on geometric data related to humans in an environment) as a function of predetermined relative hazards data corresponding to regions of the body.

In the depicted example, the communication module 330 is operably coupled to sensor(s) 410, a camera 140 (e.g., the camera 140) and orientation tags 420. The sensor(s) 410 may, for example, measure orientation and/or position of a hazards emitter. The controller 305 may, for example, receive data from the sensor(s) 410. For example, the controller 305 may determine a position of a user's body from the camera 140.

In some implementations, the controller 305 may determine a position of a user or an equipment using orientation tags 420. For example, the camera 140 may capture the orientation tags 420 and determine orientations and/or locations of objects attached to the orientation tags. The controller 305 may, for example, determine a position of a hazards emitter based on the sensor(s) 410. may determine a position of a user's body. The hazards model engine 405 may, for example, generate a visualization model at least partially as a function of user's position, the hazards emitter position, and/or predetermined physiological data (e.g., brain tissue may correspond to a higher hazard than hands).

In some implementations, the hazards model engine 405 may generate the visualization model based on an accumulative exposure of a user. In this example, the storage module 315 includes medical personnel profiles 425. For example, the medical personnel profiles 425 may include personal identification of a user (e.g., the user 105). For example, the medical personnel profiles 425 may associate an accumulated radiation exposure of a medical personnel within a period of time (e.g., a week, a month, a year).

In some embodiments, the emissions model engine 170 and/or the hazards model engine 405 may, for example, include machine learning models. For example, sensors (e.g., such as the orientation tags 420) may measure actual emissions received (e.g., radiation). The emissions model engine 170 and/or the hazards model engine 405 may train the models based on historical predicted hazards compared to historical actual emissions measured. The emissions model engine 170 and/or the hazards model engine 405 may, for example, train the models based on historical geometric data and historical measurements of actual emissions. For example, the hazards model engine 405 may generate a three dimensional model of a target environment (e.g., an operating room including objects including fixtures, wall, patient and/or medical personnel). For example, the hazards model engine 405 may determine a radiation buildup of where and/or what is hitting as time flows.

In some implementations, the hazards model engine 405 may generate a reflection percentage, absorption percentage, and/or refracted percentage when a radiation is determined to hit an object. For example, the hazards model engine 405 may identify a person using machine learning. For example, the hazard model engine 405 may identify other objects and radiation trajectory using artificial intelligence algorithms (e.g., by perceiving, synthesizing, inferring information). Accordingly, in some embodiments, the DHVS 400 may, for example, solve a technical problem of dynamically generating visual indicia of hazards based on relative positions and/or configurations (e.g., equipment type, orientation) in a dynamic (e.g., moving objects) environment.

In some embodiments, the hazards model engine 405 may use Augmented Reality library from the University of Cordoba (ARUCo) tags for training the computer vision system (e.g., an artificial intelligence model. For example, the visualization engine 145 may use key point detection to detect a position and an orientation (e.g. pose estimation) of the user 105, the emitter 120 or the radio-scattering object. In some implementations, the artificial intelligence model may receive image frames from the camera 140 and positions from the ARUCo tags. For example, the artificial intelligence model may include a recurrent convolutional neural network. For example, the artificial intelligence model may generate the key point objects with bias of ARUCo tags and poses of the user 105.

In some implementations, the artificial intelligence model may receive image frames from the camera 140. For example, based on the image frames, the artificial intelligence model may generate key points of objects in a 2-dimentional space relative to the camera 140. For example, the artificial intelligence model may also determine the pose of the user 105 (e.g., as a stick figure). For example, a radiation distribution (e.g., a typical distribution, a specialized distribution computed by the DHVS 100) may be overlayed on the image frames to be displayed. For example, the radiation distribution may be generated based on various c-arm radiography machine 110 commonly used (e.g., in Europe, in the United States, in China).

In some implementations, the hazards model engine 405 may be trained to identify the key points based on specified c-arm radiography machine 110. For example, the hazards model engine 405 may pre-train fluoroscopic specification models in various commonly used c-arm radiography machine 110. In some examples, when a position of the 110/is changed, the hazards model engine 405 may retrieve one of the pre-trained models based on detected changes. For example, the hazards model engine 405 may calibrate the retrieved model by transforming the retrieved model to match orientations and positions of the objects in a target space.

In some embodiments, for example, the display unit 355 may include one or more visualization devices. The display unit 355 may, for example, include an augmented reality (AR) display (e.g., smart glasses).

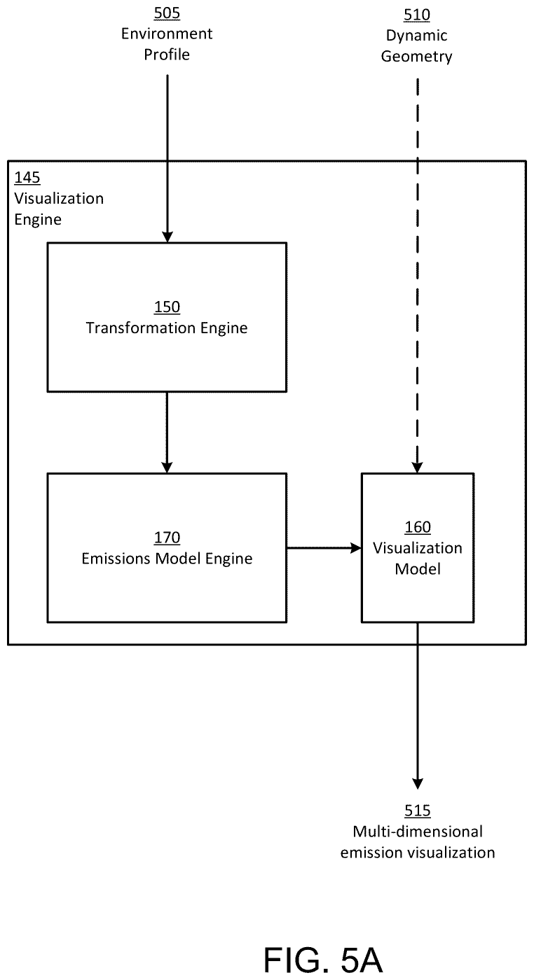

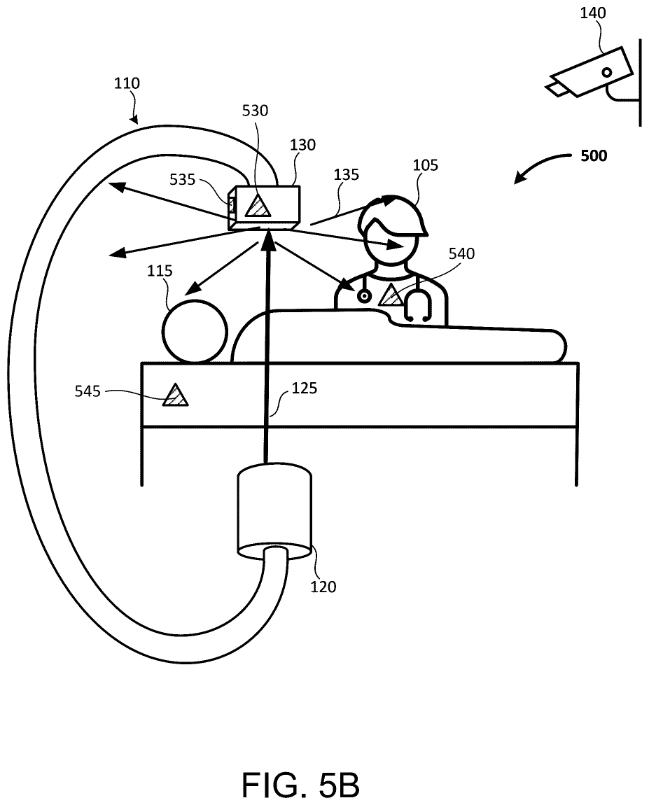

FIG. 5A depicts a block diagram of an exemplary visualization engine 145 and a visualization model 160. The visualization engine 145 includes a transformation engine 150 and an emission model engine 170. As shown, the emissions model engine 170 receives an environment profile 505 (e.g., the operation room profile 320, hazards type, equipment type, environmental configuration). For example, the environment profile 505 may be generated based on images received from the camera 140. For example, the visualization engine 145 may identify the environment profile 505 from the an image received from the camera 140. For example, the visualization engine 145 may apply computer vision model to generate the environment profile 505. For example, the transformation engine 150, using the environment profile 505 translate, rotate, scale, and/or extrapolate a selected predetermined radiation intensity model to match the geometrical relationships of objects in the environment profile 505. For example, the geometrical relationships of objects in the environment profile 505 may include a relative geometry of the radiation source and the at least one target radiation-scattering object. In some implementations, the environment profile 505 may include an Euclidean geometry of a target space (e.g., the hospital operating room 101).

The transformation engine 150 may select one of the predetermined radiation models 165 from a library from the data store 155 and generate a TRM 325. The emissions model engine 170 may, for example, generate a visualization model 160 based on the TRM 325.

In some implementations, the visualization engine 145 may receive dynamic geometry 510 (e.g., location of users). In some implementations, the visualization engine 145 may include a computer vision engine to identify and/or classify characteristics of objects in an image captured by the camera 140 to generate the dynamic geometry 510. For example, the visualization engine 145 may identify whether an object is a medical personnel or a scattering object. For example, the visualization engine 145 may identify different personnel by facial recognition. For example, the visualization engine 145 may identify an orientation of where a medical personnel is facing. For example, the visualization engine 145 may identify a position and orientation of an emission source of radiation.

By applying the dynamic geometry 510 to the visualization model 160, the visualization engine 145 may generate a multi-dimensional (e.g., 2D, 3D, 4D) emission visualization 515. In some embodiments, 4D may, for example, include three-dimensions (e.g., orthogonal dimensions such as X, Y, Z) and at least one other dimension (e.g., time). In some embodiments, the visualization engine 145 may be configured to use the visualization model to generate non-visual feedback (e.g., sound, vibration). In some embodiments, the visualization model 160 may, for example, be configured to generate non-visual feedback (e.g., sound, vibrations).

FIG. 5B depicts an exemplary embodiment including orientation tags (e.g., the orientation tags 420) configured to provide additional orientation information for generating the visualization model as described with reference to FIG. 5A. As shown in this example, an operation room 500 includes two orientation tags 530, 535 on the emitter 120 of the c-arm radiography machine 110. The scattering object, in this example, includes orientation tags 545. In the depicted example, the user 105 also wears an orientation tag 540. In some implementations, the user may, for example, not wear orientation tag 540. For example, in some implementations, user's may be automatically detected (e.g., by computer vision) without any device.

The orientation tags 530, 535, 540, 545, for example, may each include distinct predetermined patterns printed on an external surface of the tags. In some implementations, the camera 140 may, using computer vision, determine a position and an orientation of the receiver 130, the user 105, and scattering objects in the operation room 500. Accordingly, the visualization engine 145 may advantageously, in some examples, determine radiation exposure to the user dynamically based on the dynamic geometry 510 in the operation room 500 in real time.

As an illustrative example, the orientation tags 530, 535, 540, 545 may include the ARUCo tags. In some implementations, the ARUCo tags may be used to understand a position and an orientation of a c-arm radiography machine 110. For example, the visualization engine 145 may superimpose a radiation distribution based on the position and the orientation of the c-arm radiography machine 110 on an image of the hospital operating room 101. In some implementations, the visualization engine 145 may include a person identification engine (PIE). For example, the PIE may identify names and/or identification numbers, of medical personnel in the hospital operating room 101. For example, the PIE may detect a position and an orientation of each medical personnel using the ARUCo tags attached to their bodies.

In some implementations, the visualization engine 145 may generate a heat map of the radiation distribution using the detected positions and orientations of the c-arm radiography machine 110 and other radiation-scattering objects in the hospital operating room 101. For example, the heatmap may include radiation intensity in the hospital operating room 101, for example, for each person.

In some implementations, the PIE may uniquely identify each person in the hospital operating room 101. For example, the user 105 may identified based on the orientation tag 540. For example, the visualization engine 145 may use the PIE to generate a heatmap (e.g., for each person for each session) over time when the user 105 cross into the radiation distribution. Based on the heatmap, for example, the visualization engine 145 may generate specific recommendations based on predetermined factors (e.g., amount of time for a person to be in the hospital operating room 101, suggestion of mitigation strategies, correlate thermoluminescent detector (TLD) badge to actual exposure).

For example, using computer vision, the multi-dimensional emission visualization 515 may be generated based on detection of at least one of a plurality of predetermined non-electronic markers disposed in predetermined locations on the C-arm radiography machine.

In some implementations, the DHVS 100 may use the orientation tags 545 for initial setup. For example, the DHVS 100 may use radiation sensors (e.g., the sensor(s) 410) for measuring the actual radiation dosage at various points in the hospital operating room 101. For example, the DHVS 100 may use the orientation tags 545 to calibrate actual locations and orientation of the c-arm radiography machine 110 and other scattering objects. In some implementations, the DHVS 100 may generate an actual radiation model for the hospital operating room 101. In some implementations, the DHVS 100 may generate a model radiation intensity profile for the hospital operating room 101 based on the measurements from the radiation sensors.

In some implementations, when the visualization engine 145 is selecting the predetermined radiation models 165, the visualization engine 145 may generate an error attribute associated with each of the predetermined radiation models 165 in the data store 155. For example, the error attribute may include a difference between one of the predetermined radiation models 165 and the actual radiation model at various measurement points. For example, N of the predetermined radiation models 165 with the smallest error attributes may be selected.

FIG. 6 depicts an exemplary method of generating a visualization model (e.g., the visualization model 160). The method 600 may, for example, be performed by a controller (e.g., controller 305). For example, the controller 305 may use the method 600 to generate the visualization model for a target environment (e.g., the hospital operating room 101) when a user selects to start measuring the radiation data in the target environment. In this example, a signal corresponding to an environment (e.g., operating room, manufacturing floor) configuration is received in a step 605. For example, the visualization engine 145 may receive an image of an initial setup of the hospital operating room 101 from the camera 140. Next, in step 610, an environment profile of the room configuration is generated. For example, the visualization engine 145 may generate the operation room profile 320 based on images captured by the camera 140 using computer vision techniques. For example, the visualization engine 145 may generate the operation room profile 320 based on images captured by the camera 140 using computer vision techniques and the orientation tags 420.

If it is determined, in a decision point 615, that the configuration corresponds to a (predetermined/precalculated) visualization model, then a stored visualization model is retrieved in a step 620, and the method 600 ends. For example, the visualization model 160 may include metadata corresponding to a corresponding operation room profile 320 of the visualization model 160. Accordingly, the visualization engine 145 may advantageously have a faster start up time when radiation monitoring is needed. If it is determined, in the decision point 615, that the configuration does not correspond to a stored visualization model, then the environment profile is compared with profiles associated with each predetermined radiation model in a model library in step 625. For example, the transformation engine 150 may access the data store 155 to retrieve the predetermined radiation models 165. For example, the transformation engine 150 may compare the operation room profile 320 of the hospital operating room 101 with profiles associated with each of the predetermined radiation models 165.

In step 630, at least one of the predetermined radiation models is selected. For example, the transformation engine 150 may select one or more of the predetermined radiation models 165 based on the operation room profile 320. After the predetermined radiation models are selected, in step 635, the selected predetermined radiation models are transformed into a TRM. For example, the transformation engine 150 may translate, scale, and/or rotate the predetermined radiation models based on the operation room profile 320 of the hospital operating room 101. In some examples, the transformation engine 150 may also combine the more than one selected predetermined radiation models as a function of a difference of attributes between the operation room profile 320 and a model profile associated with each of the one selected predetermined radiation models.

In step 640, a visualization model is generated (e.g., by the emissions model engine 170, the hazards model engine 405). For example, the emissions model engine 170 and/or the hazards model engine 405 may, based on the TRM 325, generate a visualization model including radiation hazards models and indicia (e.g., the first visual indicium 210, second visual indicium 215, third visual indicium 220, fourth visual indicia 225). The visualization model is then stored, in a step 645, for visual generation, and the method 600 ends.

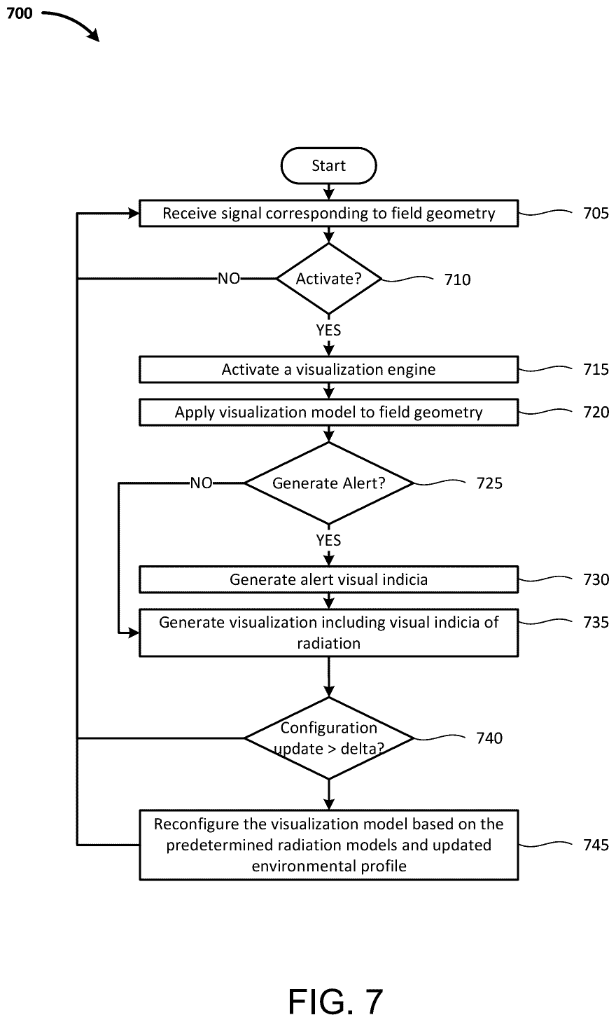

FIG. 7 depicts an exemplary method 700 of generating a visualization. In some embodiments, for example, the method 700 may be performed by a controller (e.g., controller 340). In the method 700, a signal corresponding to field geometry (e.g., a new image from the camera 140, a change in emitter orientation and/or position, user position) is received in a step 705. For example, the field geometry (e.g., the dynamic geometry 510) may be generated based on one or more images received from the camera 140. In this exemplary method, if it is determined, in the decision point 710, that the visualization should not be activated, then the method 700 returns to the step 705. For example, the visualization engine 145 may identify that no human being is present in the operation room 101. In this case, the visualization engine 145 may determine that there is no need to activate. In some examples, the visualization engine 145 may receive a user selection for activation. In some examples, the activation may be automatically turned on when a user “sign-in” (e.g., by inputting a user credential, voice activation) to the DHVS 100. For example, it may be determined whether the visualization should be activated based on whether a (hazard) emitter is on (e.g., emitting a hazard). It may be determined, for example, that the visualization should be activated based on operator input (e.g., activating a training, configuration, and/or testing mode).

If it is determined, in the decision point 710, that the visualization should be activated, then a visualization engine (e.g., the visualization engine 145) is activated in the step 715. A visualization model (e.g., predetermined, stored in the memory module 345 and/or the storage module 350, generated by the method 600) is applied to the field geometry in a step 720 to determine a corresponding visualization.

If it is determined, in the decision point 725, whether an alert is to be generated. For example, the hazards model engine 405 may determine, based on the dynamic positions and orientation of radiation sensitive objects (e.g., the user 105), whether an alert is to be generated. If it is determined that an alert is to be generated, an alert visual indicia is generated in a step 730. For example, the hazards model engine 405 may generate suggestions targeted to a particular user to avoid overexposure.

If it is determined, in the decision point 725, that an alert is not need to be generated, or once the alert visual indicia is generated in the step 730, visual indicia of radiation are generated (e.g., and emitted and/or transmitted to a display device(s)) in a step 735. For example, the visual indica 210, 215, 220, 225 may be generated according to the visualization model 160.

In a decision point 740, it is determined whether the current configuration change is bigger than a predetermined threshold. For example, the visualization engine 145 may detect that the dynamic field geometry is too different from the original configuration (e.g., from an environment profile generated in method 600). If it is determined that the dynamic field geometry is not too different, and the method 700 returns to the step 705.

If it is determined that the dynamic field geometry is too different, the visualization model is reconfigured based on the predetermined radiation models and updated environmental profile in step 745, and the method 700 returns to the step 705. For example, the method 600 may be performed.

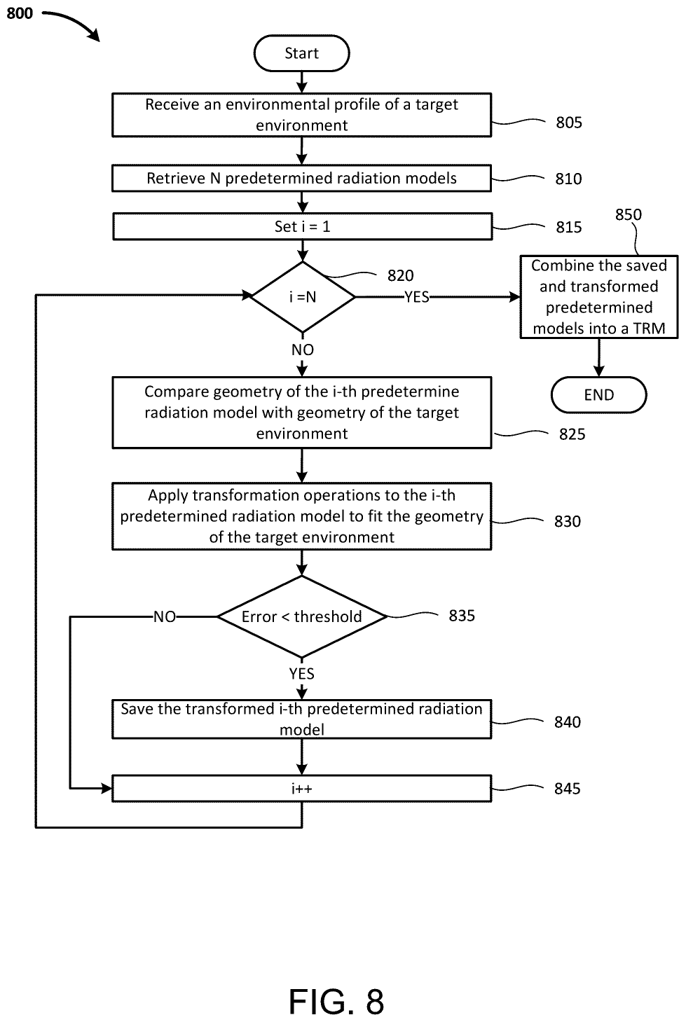

FIG. 8 depicts an exemplary method of using N predetermined emission models to generate a transformed radiation model (TRM). For example, the transformation engine 150 may be configured to perform the method 800. In this example, the method 800 begins in step 805 when an environmental profile of a target environment is received. For example, the environmental profile may be generated by the visualization engine 145 using computer vision based on images received from the camera 140. Next, N predetermined radiation models are retrieved in step 810. For example, the predetermined radiation models 165 may be retrieved from the data store 155. In step 815, a variable i is set to 1.

In a decision point 820, it is determined whether i=N. If i is not equal to N, a geometry of the i-th predetermined radiation model is compared with a geometry of the target environment in step 825. For example, the geometry of the i-th predetermined radiation model is saved in a model profile. Next, in step 830, transformation operations are applied to the i-th predetermined radiation model to fit the geometry of the target environment. For example, the transformation engine 150 may translate, scale, rotate, and extrapolate data points of the i-th predetermined radiation model to fit the geometry of the operation room profile 320.

In a decision point 835, it is determined whether an error attribute between the transformed model and the environmental profile is smaller than a threshold. For example, the transformation engine 150 may generate the error attribute as a function of a difference in geometries and/or scatter object positions and orientations.

If it is determined that the error attribute between the transformed model and the environmental profile is smaller than the threshold, the transformed i-th predetermined radiation model is saved in step 840. If it is determined that the error attribute between the transformed model and the environmental profile is greater than the threshold or after the step 840, i is set to i+1 in step 845, and the decision point 820 is repeated.

In the decision point 820, if it is determined that i=N, in step 850, the saved and transformed predetermined models are combined into a TRM. For example, the transformation engine 150 may generate the TRM based on a weighted average of the saved and transformed predetermined models, and the method 800 ends.

Although various embodiments have been described with reference to medical procedures, for example, some embodiments may be configured for general and/or non-medical applications. Some embodiments, for example, may be configured for manufacturing situations (e.g., non-destructive testing). The embodiments, for example, may be configured for gas emitting hazards. For example, a DHVS may be configured for non-emitted hazards (e.g., falling object hazards, working equipment hazards). A hazards model engine 405 may, for example, be configured to predict hazard probabilities based on impact with non-emitted hazards (e.g., traveling vehicles, robotic machinery). In some embodiments, for example, the hazards model engine 405 may be contextually adaptive, for example. For example, the hazards model engine 405 may generate visualizations based on a current environment (e.g., manufacturing, medical, indoors, outdoors, traveling) of a user. In some embodiments, the DHVS 100 may be used in a nuclear power plant. For example, the DHVS 100 may be used to monitor exposure of workers around radioactive objects.

In some embodiments, an emissions model engine (e.g., the emissions model engine 170) may, for example, generate a visualization model configured to generate a 2D intensity map visualization. The intensity map may, for example, depict statistical (historical) radiation distributions (e.g., simulations) based on a 3D model. For example, a 2D position (xᵢ, yᵢ) may, for example, be assigned a color Cᵢ corresponding to a 2D value statistical hazard intensity (e.g., relative hazard intensity) H2ᵢ. The 2D value H2ᵢ may, for example, be determined based on a (weighted) average of a 3D statistical (e.g., simulated) hazard distribution (e.g., hazard mapped to 3D volume), where a (relative) statistical hazard intensity in the 3D space may be given by H3ᵢ(xᵢ,yᵢ,zᵢ). In some embodiments, for example, H2ᵢ(Xi,yi)=max (H3ᵢ(xᵢ,yᵢ,z(0: N)), where N is a max Z value (e.g., max height). In some embodiments, H2ᵢ may, for example, be modified by a biological sensitivity weighting (e.g., a higher weighting applied to regions where simulated emissions are higher in Z values (statistically, historically) corresponding to biologically sensitive regions such as brain, reproductive organs).

Although various embodiments have been described with reference to the c-arm radiography machine 110, the DHVS may also be used to protect the user 105 from other radiation sources. For example, the DHVS 300 may include predetermined emission models for other types of radiation sources. For example, the radiation sources may include an X-ray machine. For example, the radiation sources may include a Magnetic resonance imaging (MRI) machine.

In some implementations, the emitter 120 (e.g., as described with reference to FIG. 1) of the c-arm radiography machine 110 may also be equipped with a display generator. The display generator may, for example, generate and emit an optical projection. The optical projection may, for example, be projected upwards. The optical projection may be patterned according to a visualization model. For example, the optical projection may be generated as a function of relative hazard (e.g., corresponding to (expected) concentration of scattered radiation). For example, the optical projection may create visual indicia on the floor.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) battery, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and, CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device, such as a CRT (cathode ray tube) or LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In an illustrative aspect, a dynamic hazard visualization system, may, for example, include a camera (e.g., 140). The system may, for example, include a data store (e.g., 155). The data store may, for example, include predetermined radiation intensity models (e.g., 165). The system may, for example, include a visualization engine (e.g., 145) operably coupled to the camera. The visualization engine may, for example, be configured to generate an inter-object properties data structure (e.g., 325) as a function of at least one image of a target space received from the camera and at least one of the plurality of predetermined radiation intensity models. The inter-object properties data structure may, for example, be generated by applying transformation operations to the predetermined radiation models.

The operations may, for example, include identify an environment profile (e.g., 505) of geometrical relationships of objects from the at least one image. The operations may, for example, include select at least one of the plurality of predetermined radiation intensity models based on the environment profile of geometrical relationships of objects. The operations may, for example, include transform the selected at least one predetermined radiation intensity model to match the geometrical relationships of objects in the environment profile. Transform may, for example, include at least one of: translate, rotate, scale, and extrapolate.

The system may, for example, include an emissions model engine (e.g., 170) configured to generate an instantaneous radiation intensity profile (e.g., 160) for the target space by applying a dynamic geometry (e.g., 510) to the inter-object properties data structure. The dynamic geometry may, for example, include real time geometry of at least one radiation-scattering object (e.g., 130), an orientation of a radiation source (e.g., 120), and a relative location of at least one radiation sensitive object (e.g., 105). The system may, for example, include a hazards model engine configured to generate a visualization as a function of accumulated radiation exposures of the at least one radiation-sensitive object and the dynamic geometry. The accumulated radiation exposures may, for example, be generated as a function of historical instantaneous radiation intensity profile and historical dynamic geometry. The system may, for example, include an alert generation engine (e.g., 405) configured to generate a visual indicium (e.g., 210, 215, 220, 225). The visual indicium may, for example, include real time radiation hazards (e.g., visual indication(s) of the real time radiation hazards) of the at least one radiation-sensitive object based on the instantaneous radiation intensity profile.

The at least one of the predetermined radiation intensity models may, for example, be selected to generate the inter-object properties data structure based on the environment profile of geometrical relationships of objects, such that radiation exposure of the at least one radiation-sensitive object may, for example, be determined in real time.

The geometrical relationships of objects may, for example, include a relative geometry of the radiation source and the at least one target radiation-scattering object.

The environment profile of geometrical relationship of objects may, for example, include a Euclidean geometry of the target space.

The predetermined radiation intensity models may, for example, include a predetermined visualization overlay for generating the visual indicium may, for example, include real-time radiation hazards.

The visual indicium may, for example, includes an augmented reality visualization. The augmented reality visualization may, for example, include suggested actions for the at least one radiation-sensitive object based on the instantaneous radiation intensity profile to reduce radiation exposure on the at least one radiation-sensitive object.

The system of claim 1, further may, for example, include predetermined non-electronic markers attached at least in predetermined locations on the radiation source and the at least one radiation-sensitive object. The inter-object properties data structure may, for example, be generated based on detection of at least one of the plurality of predetermined non-electronic markers.

The selection of the predetermined radiation intensity models may, for example, be determined based on selecting the predetermined radiation intensity models having a minimum error attribute. The error attribute may, for example, be generated as a function of a radiation intensity profile generated from the predetermined radiation intensity model comparing to a model radiation intensity measured using the predetermined non-electronic markers attached at least to in predetermined locations on the radiation source and the at least one radiation-sensitive object and measuring devices configured to measure an actual radiation exposure at a plurality of points of space in the environment profile.

The system further may, for example, include medical personnel profiles configured to associate a medical personnel with the accumulated radiation exposure of a medical personnel within a period of time.

In an illustrative aspect, a dynamic hazard visualization system, may, for example, include a camera (e.g., 140). The system may, for example, include a data store (e.g., 155). The data store may, for example, include predetermined radiation intensity models (e.g., 165). The system may, for example, include a visualization engine (e.g., 145) operably coupled to the camera. The visualization engine may, for example, be configured to generate an inter-object properties data structure (e.g., 325) as a function of at least one image of a target space received from the camera and at least one of the predetermined radiation intensity models. The system may, for example, include an emissions model engine (e.g., 170) configured to generate an instantaneous radiation intensity profile (e.g., 160) for the target space by applying a dynamic geometry (e.g., 510) to the inter-object properties data structure. The dynamic geometry may, for example, include, in real time, geometry of at least one radiation-scattering object (e.g., 130), an orientation of a radiation source (e.g., 120), and a relative location of at least one radiation sensitive object (e.g., 105). The system may, for example, include an alert generation engine (e.g., 405) configured to generate a visual indicium (e.g., 210, 215, 220, 225). The visual indicium may, for example, include real time radiation hazards of the at least one radiation-sensitive object based on the instantaneous radiation intensity profile. The at least one of the predetermined radiation intensity models may, for example, be selected to generate the inter-object properties data structure based on an environment profile (e.g., 505) of geometrical relationships of objects, such that radiation exposure of the at least one radiation-sensitive object may, for example, be determined in real time.

The visualization engine may, for example, be configured to generate inter-object properties data structure by applying transformation operations to the selected predetermined radiation models. The operations may, for example, include: identify the environment profile of geometrical relationship of objects from the at least one image. The operations may, for example, include transform the selected at least one predetermined radiation intensity model to match the geometrical relationships of objects in the environment profile. Transform may, for example, includes at least one of: translate, rotate, scale, and extrapolate.

The geometrical relationships of objects may, for example, include a relative geometry of the radiation source and the at least one target radiation-scattering object.

The environment profile of geometrical relationship of objects may, for example, include a Euclidean geometry of the target space.

The predetermined radiation intensity models may, for example, include a predetermined visualization overlay for generating the visual indicium. The visual indicium may, for example, include real-time radiation hazards.

The visual indicium may, for example, include an augmented reality visualization. The augmented reality visualization may, for example, include suggested actions for the at least one radiation-sensitive object based on the instantaneous radiation intensity profile to reduce radiation exposure on the at least one radiation-sensitive object.

The system further may, for example, include predetermined non-electronic markers attached at least in predetermined locations on the radiation source and the at least one radiation-sensitive object, wherein the inter-object properties data structure may, for example, be generated based on detection of at least one of the predetermined non-electronic markers.

The selection of the predetermined radiation intensity models may, for example, be determined based on selecting the predetermined radiation intensity models having a minimum error attribute. The minimum error attribute may, for example, be generated as a function of a radiation intensity profile generated from the predetermined radiation intensity model comparing to a model radiation intensity measured using the predetermined non-electronic markers attached at least to in predetermined locations on the radiation source and the at least one radiation-sensitive object and measuring devices configured to measure an actual radiation exposure at a plurality of points of space in the environment profile.

The system may, for example, include a hazards model engine configured to generate a visualization as a function of hazard levels of the at least one radiation-sensitive object and the relative geometry of the radiation source and the at least one target radiation-scattering object.

The system may, for example, include medical personnel profiles configured to associate a medical personnel with an accumulated radiation exposure of a medical personnel within a period of time.

In some implementations, operations performed by a system may, for example, be embodied in a computer program product (e.g., CPP). The CPP may, for example, include a program of instructions tangibly embodied on a non-transitory computer readable medium. When the instructions may be executed on a processor, for example, the processor may cause operations to be performed to perform dynamic hazard visualization (e.g., generating a visualization model and/or monitoring a radiation dosage of medical personnel in an operation room), such as disclosed at least with reference to FIGS. 3-5A and 6-8.

In some implementations, operations performed by a system may, for example, be implemented as a computer-implemented method. For example, the method may be performed by at least one processor. The method may include, for example, operations configured to perform dynamic hazard visualization (e.g., generating a visualization model and/or monitoring a radiation dosage of medical personnel in an operation room), such as disclosed at least with reference to FIGS. 3-5A and 6-8.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

1. A dynamic hazard visualization system, comprising:
a camera (e.g., 140);
a data store (e.g., 155) comprising a plurality of predetermined radiation intensity models (e.g., 165);
a visualization engine (e.g., 145) operably coupled to the camera, the visualization engine configured to generate an inter-object properties data structure (e.g., 325) as a function of at least one image of a target space received from the camera and at least one of the plurality of predetermined radiation intensity models, wherein the inter-object properties data structure is generated by applying transformation operations to the predetermined radiation models, wherein the operations comprise:
identify an environment profile (e.g., 505) of geometrical relationships of objects from the at least one image;
select at least one of the plurality of predetermined radiation intensity models based on the environment profile of geometrical relationships of objects; and,
transform the selected at least one predetermined radiation intensity model to match the geometrical relationships of objects in the environment profile, wherein transform comprises at least one of: translate, rotate, scale, and extrapolate;
an emissions model engine (e.g., 170) configured to generate an instantaneous radiation intensity profile (e.g., 160) for the target space by applying a dynamic geometry (e.g., 510) to the inter-object properties data structure, wherein the dynamic geometry comprises, in real time, geometry of at least one radiation-scattering object (e.g., 130), an orientation of a radiation source (e.g., 120), and a relative location of at least one radiation sensitive object (e.g., 105);
a hazards model engine configured to generate a visualization as a function of accumulated radiation exposures of the at least one radiation-sensitive object and the dynamic geometry, wherein the accumulated radiation exposures is generated as a function of historical instantaneous radiation intensity profile and historical dynamic geometry; and,
an alert generation engine (e.g., 405) configured to generate a visual indicium (e.g., 210, 215, 220, 225) comprising real time radiation hazards of the at least one radiation-sensitive object based on the instantaneous radiation intensity profile, wherein,
the at least one of the plurality of predetermined radiation intensity models are selected to generate the inter-object properties data structure based on the environment profile of geometrical relationships of objects, such that radiation exposure of the at least one radiation-sensitive object is determined in real time.

2. The dynamic hazard visualization system of claim 1, wherein the geometrical relationships of objects comprise a relative geometry of the radiation source and the at least one target radiation-scattering object.

3. The dynamic hazard visualization system of claim 1, wherein the environment profile of geometrical relationship of objects comprises a Euclidean geometry of the target space.

4. The dynamic hazard visualization system of claim 1, wherein the predetermined radiation intensity models comprise a predetermined visualization overlay for generating the visual indicium comprising real-time radiation hazards.

5. The dynamic hazard visualization system of claim 1, wherein the visual indicium comprises an augmented reality visualization, wherein the augmented reality visualization comprises suggested actions for the at least one radiation-sensitive object based on the instantaneous radiation intensity profile to reduce radiation exposure on the at least one radiation-sensitive object.

6. The dynamic hazard visualization system of claim 1, further comprising a plurality of predetermined non-electronic markers attached at least to in predetermined locations on the radiation source and the at least one radiation-sensitive object, wherein the inter-object properties data structure is generated based on detection of at least one of the plurality of predetermined non-electronic markers.

7. The dynamic hazard visualization system of claim 6, wherein the selection of the plurality of predetermined radiation intensity models is determined based on selecting the predetermined radiation intensity models having a minimum error attribute, wherein:
the error attribute is generated as a function of a radiation intensity profile generated from the predetermined radiation intensity model comparing to a model radiation intensity measured using the predetermined non-electronic markers attached at least to in predetermined locations on the radiation source and the at least one radiation-sensitive object and a plurality of measuring device configured to measure an actual radiation exposure at a plurality of points of space in the environment profile.

8. The dynamic hazard visualization system of claim 1, further comprising medical personnel profiles configured to associate a medical personnel with the accumulated radiation exposure of a medical personnel within a period of time.

9. A dynamic hazard visualization system, comprising:
a camera (e.g., 140);
a data store (e.g., 155) comprising a plurality of predetermined radiation intensity models (e.g., 165);
a visualization engine (e.g., 145) operably coupled to the camera, the visualization engine configured to generate an inter-object properties data structure (e.g., 325) as a function of at least one image of a target space received from the camera and at least one of the plurality of predetermined radiation intensity models, wherein the inter-object properties data structure is generated by applying transformation operations to the predetermined radiation models, wherein the operations comprise:
identify an environment profile (e.g., 505) of geometrical relationships of objects from the at least one image;
select at least one of the plurality of predetermined radiation intensity models based on the environment profile of geometrical relationships of objects; and,
transform the selected at least one predetermined radiation intensity model to match the geometrical relationships of objects in the environment profile;
an emissions model engine (e.g., 170) configured to generate an instantaneous radiation intensity profile (e.g., 160) for the target space by applying a dynamic geometry (e.g., 510) to the inter-object properties data structure, wherein the dynamic geometry comprises, in real time, geometry of at least one radiation-scattering object (e.g., 130), an orientation of a radiation source (e.g., 120), and a relative location of at least one radiation sensitive object (e.g., 105);
an alert generation engine (e.g., 405) configured to generate a visual indicium (e.g., 210, 215, 220, 225) comprising real time radiation hazards regions overlaying on the at least one image of the target space based on the instantaneous radiation intensity profile, wherein the real time radiation hazards regions comprise real time radiation intensity of different relative strengths, wherein,
the at least one of the plurality of predetermined radiation intensity models are selected based on an environment profile (e.g., 505) of geometrical relationships of objects to generate the inter-object properties data structure, such that radiation exposure of the at least one radiation-sensitive object is determined in real time.

10. The dynamic hazard visualization system of claim 9, wherein transform the selected at least one predetermined radiation intensity model comprises at least one of: translate, rotate, scale, and extrapolate.

11. The dynamic hazard visualization system of claim 9, wherein the geometrical relationships of objects comprise a relative geometry of the radiation source and the at least one target radiation-scattering object.

12. The dynamic hazard visualization system of claim 9, wherein the environment profile of geometrical relationship of objects comprises a Euclidean geometry of the target space.

13. The dynamic hazard visualization system of claim 9 or 21, wherein the predetermined radiation intensity models comprise a predetermined visualization overlay for generating the visual indicium comprising real-time radiation hazards.

14. The dynamic hazard visualization system of claim 9 or 21, wherein the visual indicium comprises an augmented reality visualization, wherein the augmented reality visualization comprises suggested actions for the at least one radiation-sensitive object based on the instantaneous radiation intensity profile to reduce radiation exposure on the at least one radiation-sensitive object.

15. The dynamic hazard visualization system of claim 9 or 21, further comprising a plurality of predetermined non-electronic markers attached at least to in predetermined locations on the radiation source and the at least one radiation-sensitive object, wherein the inter-object properties data structure is generated based on detection of at least one of the plurality of predetermined non-electronic markers.

16. The dynamic hazard visualization system of claim 15, wherein the selection of the plurality of predetermined radiation intensity models is determined based on selecting the predetermined radiation intensity models having a minimum error attribute, wherein:
the error attribute is generated as a function of a radiation intensity profile generated from the predetermined radiation intensity model comparing to a model radiation intensity measured using the predetermined non-electronic markers attached at least to in predetermined locations on the radiation source and the at least one radiation-sensitive object and a plurality of measuring device configured to measure an actual radiation exposure at a plurality of points of space in the environment profile.

17. The dynamic hazard visualization system of claim 9 or 21, further comprising a hazards model engine configured to generate a visualization as a function of hazard levels of the at least one radiation-sensitive object and the relative geometry of the radiation source and the at least one target radiation-scattering object.

18. The dynamic hazard visualization system of claim 9 or 21, further comprising medical personnel profiles configured to associate a medical personnel with an accumulated radiation exposure of a medical personnel within a period of time.

19. A dynamic hazard visualization system, comprising:
means for identifying a Euclidean geometry of a target space in real time;
a data store (e.g., 155) comprising a plurality of predetermined radiation intensity models (e.g., 165);
a visualization engine (e.g., 145) operably coupled to the means for identifying the Euclidean geometry of the target space, wherein the visualization engine is configured to generate an inter-object properties data structure (e.g., 325) as a function of the Euclidean geometry of the target space and at least one of the plurality of predetermined radiation intensity models, wherein the visualization engine is configured to generate the inter-object properties data structure by transforming the at least one of the plurality of predetermined radiation intensity models to match the identified Euclidean geometry, and combining the transformed predetermined radiation models;
an emissions model engine (e.g., 170) configured to generate an instantaneous radiation intensity profile (e.g., 160) by applying a dynamic geometry (e.g., 510) to the inter-object properties data structure, wherein the dynamic geometry comprises, in real time, geometry of an orientation of a radiation source (e.g., 120), at least one radiation-scattering object (e.g., 130), and a relative location of at least one radiation sensitive object (e.g., 105);
a hazards model engine (e.g., 405) configured to generate a visualization as a function of accumulated radiation exposures of the at least one radiation-sensitive object and the dynamic geometry, wherein the accumulated radiation exposures is generated as a function of historical instantaneous radiation intensity profile and historical dynamic geometry; and,
an alert generation engine (e.g., 405) configured to generate a visual indicium (e.g., 210, 215, 220, 225) comprising real time radiation hazards regions overlaying on the at least one image of the target space based on the instantaneous radiation intensity profile, wherein the real time radiation hazards regions comprise real time radiation intensity of different relative strengths, wherein,
the at least one of the plurality of predetermined radiation intensity models are selected to generate the inter-object properties data structure based on an environment profile (e.g., 505) of geometrical relationships of objects, wherein:
the selection is determined based on selecting the predetermined radiation intensity models having a minimum error attribute, wherein an error attribute is generated as a function of a radiation intensity profile generated from the predetermined radiation intensity model comparing to a model radiation intensity measured using a plurality of predetermined non-electronic markers (e.g., 545) attached at least to in predetermined locations on the radiation source and the at least one radiation-sensitive object and a plurality of measuring device (e.g., 410) configured to measure an actual radiation exposure at a plurality of points of space in the environment profile,
such that radiation exposure of the at least one radiation-sensitive object is determined in real time.

20. The dynamic hazard visualization system of claim 19, wherein the means for identifying the Euclidean geometry of the target space comprises a camera.

21. A dynamic hazard visualization system, comprising:
a camera (e.g., 140);
a data store (e.g., 155) comprising a plurality of predetermined radiation intensity models (e.g., 165);
a visualization engine (e.g., 145) operably coupled to the camera, the visualization engine configured to generate an inter-object properties data structure (e.g., 325) as a function of at least one image of a target space received from the camera and at least one of the plurality of predetermined radiation intensity models;
an emissions model engine (e.g., 170) configured to generate an instantaneous radiation intensity profile (e.g., 160) for the target space by applying a dynamic geometry (e.g., 510) to the inter-object properties data structure based on a transformation of at least one of the plurality of predetermined radiation intensity models corresponding to the dynamic geometry, wherein the dynamic geometry comprises, in real time, geometry of at least one radiation-scattering object (e.g., 130), an orientation of a radiation source (e.g., 120), and a relative location of at least one radiation sensitive object (e.g., 105);
an alert generation engine (e.g., 405) configured to generate a visual indicium (e.g., 210, 215, 220, 225) comprising real time radiation hazards regions overlaying on the at least one image of the target space based on the instantaneous radiation intensity profile, wherein the real time radiation hazards regions comprise real time radiation intensity of different relative strengths, wherein,
the at least one of the plurality of predetermined radiation intensity models are selected based on an environment profile (e.g., 505) of geometrical relationships of objects to generate the inter-object properties data structure, such that radiation exposure of the at least one radiation-sensitive object is determined in real time.
